(19) United States
(12) Patent Application Publication
(10) Pub. No.: US 2010/0210667 A1
(43) Pub. Date: Aug. 19, 2010

(54) IMIDAZO [1, 2-C] PYRIMIDIN-2-YLMETHYLPIPERIDINES AS OREXIN RECEPTOR ANTAGONISTS

(76) Inventors: Giuseppe Alvaro, Verona (IT); David Amantini, Verona (IT); Sandro Belvedere, Verona (IT)

Correspondence Address: GlaxoSmithKline;GLOBAL PATENTS -US, UW2220, P. O. BOX 1539, KING OF PRUSSIA, PA, 19406-0939, US

(21) Appl. No.: 12/665,787

(22) PCT Filed: Jul. 1, 2008
(86) PCT No.: PCT/EP08/58428

§ 371 Date: Dec. 21, 2009

(30) Foreign Application Priority Data
Jul. 3, 2007 (GB) 0712888.7

(51) Int. Cl. A61K 31/519; C07D 487/04; A61P 25/24; A61P 25/22; A61P 25/30; A61P 11/00; A61P 9/00

(52) U.S. Cl. 514/259.5; 544/281; 514/259.1

(57) ABSTRACT

Disclosed are imidazo[1,2-c]pyrimidin-2-ylmethyl substituted piperidine derivatives having the formula:

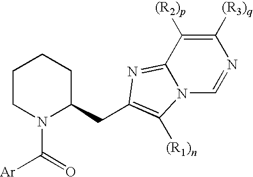

where Ar is

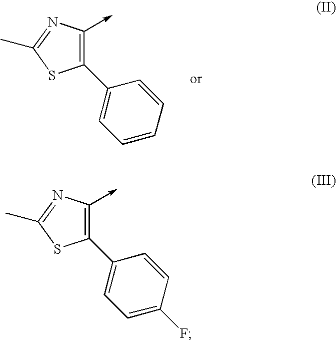

and where R₁, R₂, R₃, n, p and q are as defined herein, and their use as pharmaceuticals.

This invention relates to imidazo[1,2-c]pyrimidin-2-ylmethyl substituted piperidine derivatives and their use as pharmaceuticals.

Many medically significant biological processes are mediated by proteins participating in signal transduction pathways that involve G-proteins and/or second messengers.

Polypeptides and polynucleotides encoding the human 7-transmembrane G-protein coupled neuropeptide receptor, orexin-1 (HFGAN72), have been identified and are disclosed in EP875565, EP875566 and WO 96/34877. Polypeptides and polynucleotides encoding a second human orexin receptor, orexin-2 (HFGANP), have been identified and are disclosed in EP893498.

Polypeptides and polynucleotides encoding polypeptides which are ligands for the orexin-1 receptor, e.g. orexin-A (Lig72A) are disclosed in EP849361.

The orexin ligand and receptor system has been well characterised since its discovery (see for example Sakurai, T. et al (1998) Cell, 92 pp 573 to 585; Smart et al (1999) British Journal of Pharmacology 128 pp 1 to 3; Willie et al (2001) Ann. Rev. Neurosciences 24 pp 429 to 458; Sakurai (2007) Nature Reviews Neuroscience 8 pp 171 to 181; Ohno and Sakurai (2008) Front. Neuroendocrinology 29 pp 70 to 87). From these studies it has become clear that orexins and orexin receptors play a number of important physiological roles in mammals and open up the possibility of the development of new therapeutic treatments for a variety of diseases and disorders as described hereinbelow.

Experiments have shown that central administration of the ligand orexin-A stimulated food intake in freely-feeding rats during a 4 hour time period. This increase was approximately four-fold over control rats receiving vehicle. These data suggest that orexin-A may be an endogenous regulator of appetite (Sakurai, T. et al (1998) Cell, 92 pp 573 to 585; Peyron et al (1998) J. Neurosciences 18 pp 9996 to 10015; Willie et al (2001) Ann. Rev. Neurosciences 24 pp 429 to 458). Therefore, antagonists of the orexin-A receptor(s) may be useful in the treatment of obesity and diabetes. In support of this it has been shown that orexin receptor antagonist SB334867 potently reduced hedonic eating in rats (White et al (2005) Peptides 26 pp 2231 to 2238) and also attenuated high-fat pellet self-administration in rats (Nair et al (2008) British Journal of Pharmacology, published online 28 Jan. 2008). The search for new therapies to treat obesity and other eating disorders is an important challenge. According to WHO definitions a mean of 35% of subjects in 39 studies were overweight and a further 22% clinically obese in westernised societies. It has been estimated that 5.7% of all healthcare costs in the USA are a consequence of obesity. About 85% of Type 2 diabetics are obese. Diet and exercise are of value in all diabetics. The incidence of diagnosed diabetes in westernised countries is typically 5% and there are estimated to be an equal number undiagnosed. The incidence of both diseases is rising, demonstrating the inadequacy of current treatments which may be either ineffective or have toxicity risks including cardiovascular effects. Treatment of diabetes with sulfonylureas or insulin can cause hypoglycaemia, whilst metformin causes GI side-effects. No drug treatment for Type 2 diabetes has been shown to reduce the long-term complications of the disease. Insulin sensitisers will be useful for many diabetics, however they do not have an anti-obesity effect.

As well as having a role in food intake, the orexin system is also involved in sleep and wakefulness. Rat sleep/EEG studies have shown that central administration of orexin-A, an agonist of the orexin receptors, causes a dose-related increase in arousal, largely at the expense of a reduction in paradoxical sleep and slow wave sleep 2, when administered at the onset of the normal sleep period (Hagan et al (1999) Proc. Natl. Acad. Sci. 96 pp 10911 to 10916). The role of the orexin system in sleep and wakefulness is now well established (Sakurai (2007) Nature Reviews Neuroscience 8 pp 171 to 181; Ohno and Sakurai (2008) Front. Neuroendocrinology 29 pp 70 to 87; Chemelli et al (1999) Cell 98 pp 437 to 451; Lee et al (2005) J. Neuroscience 25 pp 6716 to 6720; Piper et al (2000) European J Neuroscience 12 pp 726-730 and Smart and Jerman (2002) Pharmacology and Therapeutics 94 pp 51 to 61). Antagonists of the orexin receptors may therefore be useful in the treatment of sleep disorders including insomnia. Studies with orexin receptor antagonists, for example SB334867, in rats (see for example Smith et al (2003) Neuroscience Letters 341 pp 256 to 258) and more recently dogs and humans (Brisbare-Roch et al (2007) Nature Medicine 13(2) pp 150 to 155) further support this.

In addition, recent studies have suggested a role for orexin antagonists in the treatment of motivational disorders, such as disorders related to reward seeking behaviours for example drug addiction and substance abuse (Borgland et al (2006) Neuron 49(4) pp 589-601; Boutrel et al (2005) Proc. Natl. Acad. Sci. 102(52) pp 19168 to 19173; Harris et al (2005) Nature 437 pp 556 to 559).

International Patent Applications WO99/09024, WO99/58533, WO00/47577 and WO00/47580 disclose phenyl urea derivatives and WO00/47576 discloses quinolinyl cinnamide derivatives as orexin receptor antagonists. WO05/118548 discloses substituted 1,2,3,4-tetrahydroisoquinoline derivatives as orexin antagonists.

WO01/96302, WO02/44172, WO02/89800, WO03/002559, WO03/002561, WO03/032991, WO03/037847, WO03/041711 and WO08/038251 all disclose cyclic amine derivatives.

WO03/002561 discloses N-aroyl cyclic amine derivatives as orexin antagonists. Compounds disclosed in WO03/002561 include piperidine derivatives substituted at the 2-position with bicyclic heteroarylmethyl groups. We have now unexpectedly found that some piperidine derivatives substituted at the 2- position with an imidazo[1,2-c]pyrimidin-2-ylmethyl group have surprisingly beneficial properties including, for example, increased oral bioavailability and significantly increased solubility in physiologically relevant media compared to the prior art compounds. Such properties make these imidazo[1,2-c]pyrimidin-2-ylmethyl substituted piperidine derivatives very attractive as potential pharmaceutical agents which may be useful in the prevention or treatment of obesity, including obesity observed in Type 2 (non-insulin-dependent) diabetes patients, sleep disorders, anxiety, depression, schizophrenia, drug dependency or compulsive behaviour. Additionally these compounds may be useful in the treatment of stroke, particularly ischemic or haemorrhagic stroke, and/or blocking the emetic response, i.e. useful in the treatment of nausea and vomiting.

Accordingly the present invention provides a compound of formula (I)

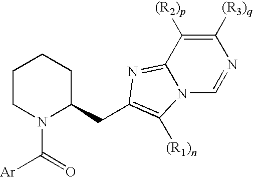

where Ar is selected from the group consisting of formula:

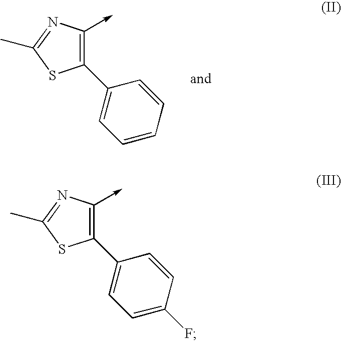

R₁ is (C₁₋₄)alkyl, halo, halo(C₁₋₄)alkyl, (C₁₋₄)alkoxy, halo(C₁₋₄)alkoxy, (C₁₋₄)alkyl-O-(C₁₋₄)alkyl, CN, NR⁴R⁵ wherein R⁴ is H or (C₁₋₄)alkyl and R⁵ is H or (C₁₋₄)alkyl; R₂ is (C₁₋₄)alkyl, halo, halo(C₁₋₄)alkyl, (C₁₋₄)alkoxy, halo(C₁₋₄)alkoxy, (C₁₋₄)alkyl-O-(C₁₋₄)alkyl, CN, NR⁶R⁷ wherein R⁶ is H or (C₁₋₄)-alkyl and R⁷ is H or (C₁₋₄)-alkyl; R₃ is (C₁₋₄)alkyl, halo, halo(C₁₋₄)alkyl, (C₁₋₄)alkoxy, halo(C₁₋₄)alkoxy, (C₁₋₄)alkyl-O-(C₁₋₄)alkyl, CN, NR⁸R⁹ wherein R⁸ is H or (C₁₋₄)-alkyl and R⁹ is H or (C₁₋₄)-alkyl;
n is 0 or 1;
p is 0 or 1; and
q is 0 or 1;
with the proviso that p and q are not both 0;
or a pharmaceutically acceptable salt thereof.

In one embodiment Ar is a group of formula (II).

In another embodiment Ar is a group of formula (III).

In one embodiment Ar is a group of formula (II) and n is 0.

In another embodiment Ar is a group of formula (II), n is 0, p is 1, q is 0 and R₂ is methyl.

In one embodiment Ar is a group of formula (II), n is 0, p is 1, q is 1 and one of R₂ and R₃ is halo and the other is (C₁₋₄)-alkyl.

In one embodiment Ar is a group of formula (II), n is 0, p is 1, q is 1, R₂ is (C₁₋₄)-alkyl and R₃ is halo.

In another embodiment Ar is a group of formula (II), n is 0, p is 1, q is 1, R₂ is methyl and R₃ is chloro.

In one embodiment Ar is a group of formula (II), n is 0, p is 1, q is 1, R₂ is halo and R₃ is (C₁₋₄)-alkyl.

In another embodiment Ar is a group of formula (II), n is 0, p is 1, q is 1, R₂ is chloro and R₃ is methyl.

In one embodiment Ar is a group of formula (II), n is 0, p is 1, q is 1 and one of R₂ and R₃ is (C₁₋₄)alkoxy and the other is (C₁₋₄)-alkyl.

In one embodiment Ar is a group of formula (II), n is 0, p is 1, q is 1, R₂ is (C₁₋₄)-alkyl and R₃ is (C₁₋₄)alkoxy.

In another embodiment Ar is a group of formula (II), n is 0, p is 1, q is 1, R₂ is methyl and R₃ is methyloxy.

In one embodiment Ar is a group of formula (III) and n is 0.

In another embodiment Ar is a group of formula (III), n is 0, p is 1, q is 0 and R₂ is methyl.

In one embodiment Ar is a group of formula (III), n is 0, p is 1, q is 1 and one of R₂ and R₃ is halo and the other is (C₁₋₄)-alkyl.

In one embodiment Ar is a group of formula (III), n is 0, p is 1, q is 1, R₂ is (C₁₋₄)-alkyl and R₃ is halo.

In another embodiment Ar is a group of formula (III), n is 0, p is 1, q is 1, R₂ is methyl and R₃ is chloro.

In one embodiment Ar is a group of formula (III), n is 0, p is 1, q is 1, R₂ is halo and R₃ is (C₁₋₄)-alkyl.

In another embodiment Ar is a group of formula (III), n is 0, p is 1, q is 1, R₂ is chloro and R₃ is methyl.

In one embodiment Ar is a group of formula (III), n is 0, p is 1, q is 1 and one of R₂ and R₃ is (C₁₋₄)alkoxy and the other is (C₁₋₄)-alkyl.

In one embodiment Ar is a group of formula (III), n is 0, p is 1, q is 1, R₂ is (C₁₋₄)-alkyl and R₃ is (C₁₋₄)alkoxy.

In another embodiment Ar is a group of formula (III), n is 0, p is 1, q is 1, R₂ is methyl and R₃ is methyloxy.

When the compound contains a (C₁₋₄)alkyl group, whether alone or forming part of a larger group, e.g. (C₁₋₄)alkoxy, the alkyl group may be straight chain, branched or cyclic, or combinations thereof. Examples of (C₁₋₄)alkyl are methyl or ethyl. An example of (C₁₋₄)alkoxy is methyloxy.

Examples of halo(C₁₋₄)alkyl include trifluoromethyl (i.e. —CF₃).

Examples of (C₁₋₄)alkoxy include methyloxy and ethyloxy.

Examples of halo(C₁₋₄)alkoxy include trifluoromethyloxy (i.e. —OCF₃).

Examples of (C₂₋₄)alkenyl include ethenyl.

Examples of HO(C₁₋₄)alkyl include hydroxymethyl.

Halogen or “halo” (when used, for example, in halo(C₁₋₄)alkyl) means fluoro, chloro, bromo or iodo.

It is to be understood that the present invention covers all combinations of particularised groups and substituents described herein above.

In one embodiment the invention provides the compound of formula (I) selected from the group consisting of:

7-chloro-8-methyl-2-({(2S)-1-[(2-methyl-5-phenyl-1,3-thiazol-4-yl)carbonyl]-2-piperidinyl}methyl)imidazo[1,2-c]pyrimidine;

8-methyl-2-({(2S)-1-[(2-methyl-5-phenyl-1,3-thiazol-4-yl)carbonyl]-2-piperidinyl}methyl)imidazo[1,2-c]pyrimidine;

8-chloro-7-methyl-2-({(2S)-1-[(2-methyl-5-phenyl-1,3-thiazol-4-yl)carbonyl]-2-piperidinyl}methyl)imidazo[1,2-c]pyrimidine;

8-methyl-7-(methyloxy)-2-({(2S)-1-[(2-methyl-5-phenyl-1,3-thiazol-4-yl)carbonyl]-2-piperidinyl}methyl)imidazo[1,2-c]pyrimidine; and

7,8-dimethyl-2-({(2S)-1-[(2-methyl-5-phenyl-1,3-thiazol-4-yl)carbonyl]-2-piperidinyl}methyl)imidazo[1,2-c]pyrimidine,

or a pharmaceutically acceptable salt thereof.

It will be appreciated that for use in medicine the salts of the compounds of formula (I) should be pharmaceutically acceptable. Suitable pharmaceutically acceptable salts will be apparent to those skilled in the art. Pharmaceutically acceptable salts include those described by Berge, Bighley and Monkhouse J.Pharm.Sci (1977) 66, pp 1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention.

Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form and, if crystalline, may optionally be solvated, eg. as the hydrate. This invention includes within its scope stoichiometric solvates (eg. hydrates) as well as compounds containing variable amounts of solvent (eg. water).

It will be understood that the invention includes pharmaceutically acceptable derivatives of compounds of formula (I) and that these are included within the scope of the invention.

As used herein “pharmaceutically acceptable derivative” includes any pharmaceutically acceptable ester or salt of such ester of a compound of formula (I) which, upon administration to the recipient is capable of providing (directly or indirectly) a compound of formula (I) or an active metabolite or residue thereof.

The compounds of formula (I) are S enantiomers. Where additional chiral centres are present in compounds of formula (I), the present invention includes within its scope all possible enantiomers and diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms or mixtures thereof.

The subject invention also includes isotopically-labeled compounds which are identical to those recited in formula (I) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or 125I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, ie. ³H, and carbon-14, ie. ¹⁴C, isotopes are particularly preferred for their ease of preparation and detectability. ¹¹C and ¹⁸F isotopes are particularly useful in PET (positron emission tomography).

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further aspect of the present invention there is provided a process for the preparation of compounds of formula (I) and derivatives thereof. The following schemes detail some synthetic routes to compounds of the invention. In the following schemes reactive groups can be protected with protecting groups and deprotected according to well established techniques.

### Schemes

According to a further feature of the invention there is provided a process for the preparation of compounds of formula (I) or salts thereof. The following is an example of a synthetic scheme that may be used to synthesise the compounds of the invention.

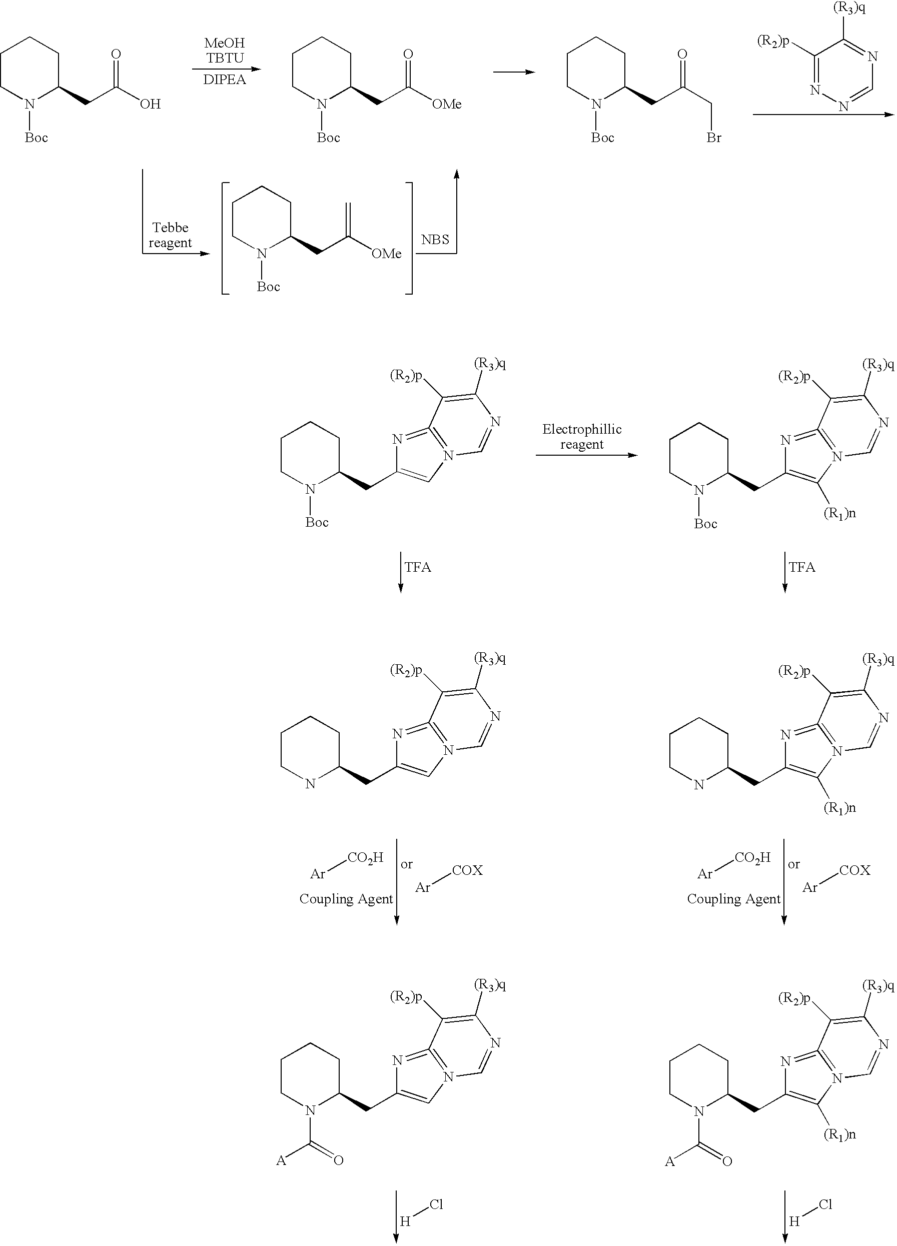

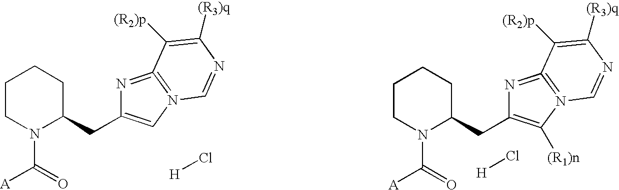

It will be understood by those skilled in the art that certain compounds of the invention can be converted into other compounds of the invention according to standard chemical methods.

The starting materials for use in the scheme are commercially available, known in the literature or can be prepared by known methods. The preparation of 5-phenyl-2-methyl-1,3-thiazole-4-carboxylic acids (the Ar groups) has been described in, for example, Mamedov et al (1991) Izvestiya Akademii Nauk SSSR, Seriya Khimicheskaya 12 pp 2832-2836. Mamedov et al (2004) Russian Journal of Organic Chemistry (Translation of Zhurnal Organicheskoi Khimii) 40(4) pp 534-542. ((2S)-1-{[(1,1-dimethylethyl)oxy]carbonyl}-2-piperidinyl)acetic acid is available from Neosystem Product List (BA19302).

Pharmaceutically acceptable salts may be prepared conventionally by reaction with the appropriate acid or acid derivative.

The present invention provides compounds of formula (I) or a pharmaceutically acceptable salt thereof for use in human or veterinary medicine.

The compounds of formula (I) or their pharmaceutically acceptable salts may be of use for the treatment or prophylaxis of a disease or disorder where an antagonist of a human orexin receptor is required such as sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

In addition the compounds of formula (I) or their pharmaceutically acceptable salts may be of use for the treatment or prophylaxis of a disease or disorder where an antagonist of a human orexin receptor is required such as depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

Further, the compounds of formula (I) or their pharmaceutically acceptable salts may be of use for the treatment or prophylaxis of a disease or disorder where an antagonist of a human orexin receptor is required such as anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00).

In addition the compounds of formula (I) or their pharmaceutically acceptable salts may be of use for the treatment or prophylaxis of a disease or disorder where an antagonist of a human orexin receptor is required such as substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic- Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

In addition the compounds of formula (I) or their pharmaceutically acceptable salts may be of use for the treatment or prophylaxis of a disease or disorder where an antagonist of a human orexin receptor is required such as feeding disorders such as bulimia nervosa, binge eating, obesity, including obesity observed in Type 2 (non-insulin-dependent) diabetes patients. Further, the compounds of formula (I) or their pharmaceutically acceptable salts may be of use for the treatment or prophylaxis of a disease or disorder where an antagonist of a human orexin receptor is required such as stroke, particularly ischemic or haemorrhagic and/or in blocking an emetic response i.e. nausea and vomiting.

The numbers in brackets after the listed diseases refer to the classification code in DSM-IV: Diagnostic and Statistical Manual of Mental Disorders, 4th Edition, published by the American Psychiatric Association. The various subtypes of the disorders mentioned herein are contemplated as part of the present invention.

The invention also provides a method of treating or preventing a disease or disorder where an antagonist of a human orexin receptor is required, for example those diseases and disorders mentioned hereinabove, which comprises administering to a subject in need thereof an effective amount of a compound of formula (I) or a pharmaceutically acceptable salt thereof.

The invention also provides a compound of formula (I), or a pharmaceutically acceptable salt thereof, for use in the treatment or prophylaxis of a disease or disorder where an antagonist of a human orexin receptor is required, for example those diseases and disorders mentioned hereinabove.

The invention also provides the use of a compound of formula (I), or a pharmaceutically acceptable salt thereof, in the manufacture of a medicament for the treatment or prophylaxis of a disease or disorder where an antagonist of a human Orexin receptor is required, for example those diseases and disorders mentioned hereinabove.

For use in therapy the compounds of the invention are usually administered as a pharmaceutical composition. The invention also provides a pharmaceutical composition comprising a compound of formula (I), or a pharmaceutically acceptable salt thereof, and a pharmaceutically acceptable carrier.

The compounds of formula (I) or their pharmaceutically acceptable salts may be administered by any convenient method, e.g. by oral, parenteral, buccal, sublingual, nasal, rectal or transdermal administration, and the pharmaceutical compositions adapted accordingly.

The compounds of formula (I) or their pharmaceutically acceptable salts which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

In one embodiment the composition is in unit dose form such as a tablet, capsule or ampoule.

The dose of the compound of formula (I), or a pharmaceutically acceptable salt thereof, used in the treatment or prophylaxis of the abovementioned disorders or diseases will vary in the usual way with the particular disorder or disease being treated, the weight of the subject and other similar factors. However, as a general rule, suitable unit doses may be 0.05 to 1000 mg, more suitably 0.05 to 500 mg. Unit doses may be administered more than once a day for example two or three times a day, so that the total daily dosage is in the range of about 0.01 to 100 mg/kg. Such therapy may extend for a number of weeks or months. In the case of pharmaceutically acceptable derivatives the above figures are calculated as the parent compound of formula (I).

Orexin-A (Sakurai, T. et al (1998) Cell, 92 pp 573-585) can be employed in screening procedures for compounds which inhibit the ligand's activation of the orexin-1 or orexin-2 receptors.

In general, such screening procedures involve providing appropriate cells which express the orexin-1 or orexin-2 receptor on their surface. Such cells include cells from mammals, yeast, Drosophila or E. coli. In particular, a polynucleotide encoding the orexin-1 or orexin-2 receptor is used to transfect cells to express the receptor. The expressed receptor is then contacted with a test compound and an orexin-1 or orexin-2 receptor ligand, as appropriate, to observe inhibition of a functional response. One such screening procedure involves the use of melanophores which are transfected to express the orexin-1 or orexin-2 receptor, as described in WO 92/01810.

Another screening procedure involves introducing RNA encoding the orexin-1 or orexin-2 receptor into Xenopus oocytes to transiently express the receptor. The receptor oocytes are then contacted with a receptor ligand and a test compound, followed by detection of inhibition of a signal in the case of screening for compounds which are thought to inhibit activation of the receptor by the ligand.

Another method involves screening for compounds which inhibit activation of the receptor by determining inhibition of binding of a labelled orexin-1 or orexin-2 receptor ligand to cells which have the orexin-1 or orexin-2 receptor (as appropriate) on their surface. This method involves transfecting a eukaryotic cell with DNA encoding the orexin-1 or orexin-2 receptor such that the cell expresses the receptor on its surface and contacting the cell or cell membrane preparation with a compound in the presence of a labelled form of an orexin-1 or orexin-2 receptor ligand. The ligand may contain a radioactive label. The amount of labelled ligand bound to the receptors is measured, e.g. by measuring radioactivity.

Yet another screening technique involves the use of FLIPR equipment for high throughput screening of test compounds that inhibit mobilisation of intracellular calcium ions, or other ions, by affecting the interaction of an orexin-1 or orexin-2 receptor ligand with the orexin-1 or orexin-2 receptor as appropriate.

Throughout the specification and claims which follow, unless the context requires otherwise, the word ‘comprise’, and variations such as ‘comprises’ and ‘comprising’ will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The following Examples illustrate the preparation of certain compounds of formula (I) or salts thereof. The Descriptions 1 to 10 illustrate the preparation of intermediates used to make compounds of formula (I) or salts thereof.

In the procedures that follow, after each starting material, reference to a description is typically provided. This is provided merely for assistance to the skilled chemist. The starting material may not necessarily have been prepared from the Description referred to.

The yields were calculated assuming that products were 100% pure if not stated otherwise.

The compounds described in the Examples described hereinafter have all been prepared as a first step from stereochemically pure ((2S)-1-{[(1,1-dimethylethyl)oxy]carbonyl}-2-piperidinyl)acetic acid. The stereochemistry of the compounds of the Descriptions and Examples have been assigned on the assumption that the pure configuration is maintained.

Compounds are named using ACD/Name PRO 6.02 chemical naming software (Advanced Chemistry Development Inc., Toronto, Ontario, M5H2L3, Canada).

Proton Magnetic Resonance (NMR) spectra were recorded either on Varian instruments at 400, 500 or 600 MHz, or on a Bruker instrument at 400 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is usually reported.

HPLC analyses indicated by HPLC (walk-up): rt=x min, were performed on a Agilent 1100 series instrument using a Luna 3u C18(2) 100A column (50×2.0 mm, 3 μm particle size) [Mobile phase and Gradient: 100% (water+0.05% TFA) to 95% (acetonitrile+0.05% TFA) in 8 min. Column T=40 ° C. Flow rate=1 mL/min. UV detection wavelength=220 nm].

Direct infusion Mass spectra (MS) were run on a Agilent MSD 1100 Mass Spectrometer, operating in ES (+) and ES (−) ionization mode [ES (+): Mass range: 100-1000 amu. Infusion solvent: water +0.1% HCO₂H/CH₃CN 50/50. ES (−): Mass range: 100-1000 amu. Infusion solvent: water +0.05% NH₄OH/CH₃CN 50/50].

Total ion current (TIC) and DAD UV chromatographic traces together with MS and UV spectra associated with the peaks were taken on a UPLC/MS AcquityTM system equipped with 2996 PDA detector and coupled to a Waters Micromass ZQTM mass spectrometer operating in positive or negative electrospray ionisation mode [LC/MS−ES (+ or −): analyses performed using an AcquityTM UPLC BEH C18 column (50×2.1 mm, 1.7 μm particle size). Mobile phase: A−water+0.1% HCO₂H/B−CH₃CN+0.06% HCO₂H. Gradient: t=0 min 3% B, t=0.05 min 6% B, t=0.57 min 70% B, t=1.06 min 99% B lasting for 0.389 min, t=1.45 min 3% B, stop time 1.5 min. Column T=40° C. Flow rate=1.0 mL/min. Mass range: ES (+): 100-1000 amu. ES (−): 100-800 amu. UV detection range: 210-350 nm. The usage of this methodology is indicated by “UPLC” in the analytic characterization of the described compounds.

For reactions involving microwave irradiation, a Personal Chemistry Emrys™ Optimizer was used.

In a number of preparations, purification was performed using Biotage manual flash chromatography (Flash+), Biotage automatic flash chromatography (Horizon, SP1 and SP4), Flash Master Personal or Vac Master systems.

Flash chromatography was carried out on silica gel 230-400 mesh (supplied by Merck AG Darmstadt, Germany), Varian Mega Be-Si pre-packed cartridges, pre-packed Biotage silica cartridges (e.g. Biotage SNAP cartridge) or KP-NH prepacked flash cartridges.

SPE-SCX cartridges are ion exchange solid phase extraction columns supplied by Varian. The eluent used with SPE-SCX cartridges is methanol followed by 2N ammonia solution in methanol.

SPE-Si cartridges are silica solid phase extraction columns supplied by Varian.

The following table lists the used abbreviations:

| | |
|---|---|
| BINAP | 2,2′-Bis(diphenylphosphino)-1,1′-binaphthyl |
| Boc | t-Butoxycarbonyl |
| t-Bu | t-Butyl |
| Cy | Cyclohexanes |
| DBA | Dibenzyilidene acetone |
| DCM | Dichloromethane |
| DIAD | Diisopropyl azodicarboxylate |
| DIPEA | N,N-diisopropyl-N-ethylamine |
| DMF | N,N-Dimethylformamide |
| Et₂O | Diethylether |
| EtOAc | Ethylacetate |
| MeOH | Methanol |
| NBS | N-Bromosuccinimide |
| rt | Retention time |
| T | Temperature |
| TBTU | O-(benzotriazol-1-yl)-N,N,N′N′-tetramethyluronium |
| | tetrafluoroborate |
| TEA | Triethylamine |
| TFA | Trifluoroacetic acid |
| THF | Tetrahydrofuran |

### DESCRIPTIONS

### Description 1

### 1,1-dimethylethyl (2S)-2-[2-(methyloxy)-2-oxoethyl]-1-piperidinecarboxylate (D1)

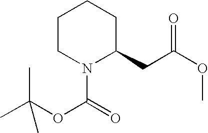

A mixture of ((2S)-1-{[(1,1-dimethylethyl)oxy]carbonyl}-2-piperidinyl)acetic acid (1 g, 4.11 mmol), DIPEA (2.15 ml, 12.33 mmol) and TBTU (1.98 g, 6.17 mmol) in DMF (25 ml) was stirred at room temperature for 20 min and the colour of the mixture darkened. MeOH (0.25 ml, 6.17 mmol) was added and the resulting reaction mixture stirred at room temperature for 30 min. The mixture was transferred into a separatory funnel containing brine (20 ml) and extracted with EtOAc (2×20 ml). The combined organic layers were washed with water/ice (5×20 ml). The organic layer was dried (Na₂SO₄), filtered and concentrated. The residue was purified by flash chromatography on silica gel (Biotage SP1, from Cy 100 to Cy/EtOAc 85/15). Collected fractions gave the title compound D1 (1.01 g, 3.92 mmol, 95% yield) as a colorless oil. ¹H-NMR (400 MHz, CDCl₃) δ(ppm): 4.67-4.75 (m, 1 H), 3.96-4.05 (m, 1 H), 3.67 (s, 3 H), 2.79 (t, 1 H), 2.61 (dd, 1H), 2.53 (dd, 1 H), 1.60-1.70 (m, 6 H), 1.46 (s, 9 H).

### Description 2

### 1,1-dimethylethyl (2S)-2-(3-bromo-2-oxopropyl)-1-piperidinecarboxylate (D2)

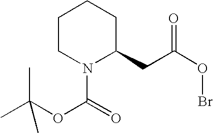

### Preparation (i)

In a 500 ml round-bottom flask under nitrogen at room temperature, 1,1-dimethylethyl (2S)-2[2-(methyloxy)-2-oxoethyl]-1-piperidinecarboxylate D1 (11.10 g, 43.10 mmol) was dissolved in THF (100 ml) to give a pale yellow solution. This solution was cooled to −78° C. and the Tebbe reagent (104 ml of a 0.5 M solution in toluene, 51.80 mmol) was added dropwise. The thick mixture was diluted with further 70 ml of dry toluene. The resulting brown-orange mixture was stirred at −78° C. for 30 min and then slowly warmed up to room temperature and left under stirring for 2 h. The reaction mixture was charged into a dropping funnel and then added dropwise to a 2 L round-bottom flask containing about 400 ml of an ice-cooled 1 M NaOH aqueous solution. At the end of the quench, the resulting grey suspension was diluted with EtOAc (250 ml) and allowed to stir overnight. The resulting yellow suspension was then filtered over a Gooch funnel and salts were washed with EtOAc (500 ml). Phases were then separated and the organic layer was washed with brine (2×500 ml). The organic phase was dried (Na₂SO₄), filtered and concentrated to give a deep orange oil. The residue was diluted with Et₂O (about 500 ml). Some salts precipitated and the resulting suspension was filtered over a Gooch funnel. The filtrate was concentrated under vacuum to give 12.40 g of 1,1-dimethylethyl (25)-2-[2-(methyloxy)-2-propen-1-yl]-1-piperidinecarboxylate as an orange-brown crude oil. The material contained some residual salts (the overall recovered amount was higher than the theoretical amount). The material was used without further purification in the next reaction and supposed to be pure at 88.7 wt %. In a 1 L round-bottom flask under nitrogen at room temperature 1,1-dimethylethyl (2S)-2-[2-(methyloxy)-2-propen-1-yl]-1-piperidinecarboxylate (12.40 g, 43.10 mmol) was dissolved in THF (125 ml) and water (35 ml) to give a pale yellow solution. NBS (7.67 g, 43.10 mmol) was then added dissolved in about 100 ml of THF. The resulting grey mixture was stirred at room temperature for 1 h. Additional NBS (1.50 g, 0.2 eq) dissolved in 50 ml of THF was added and the reaction mixture stirred at room temperature for 1 h. The mixture was concentrated under vacuum to remove THF, then was diluted with EtOAc (about 500 ml) and water (200 ml). Phases were separated and the aqueous layer was back-extracted with EtOAc (250 ml). The combined organic layers were dried (Na₂SO₄), filtered and concentrated to give 17.80 g of a brown oil. The material was purified by flash chromatography on silica gel (Biotage 75L, Cy/EtOAc from 100/0 to 90/10) to give the title compound D2 (6.00 g, 18.70 mmol, 43.5% yield from D1, two steps) as a yellow oil.

UPLC: rt=0.79 min, peaks observed: 342 (M+Na, 100%) and 344 (M+Na, 100%), 264 (M-tBu, 100%) and 266 (M-tBu, 100%). C₁₃H₂₂BrNO₃ requires 319.

¹H NMR (400 MHz, CDCl₃) δ(ppm): 4.72-4.79 (m, 1 H), 3.91-4.10 (m, 3 H), 2.77-2.97 (m, 3 H), 1.49-1.75 (m, 6 H), 1.46 (s, 9 H).

### Alternative Preparation (ii)

An alternative route to (1,1-dimethylethyl (2S)-2-(3-bromo-2-oxopropyl)-1-piperidinecarboxylate) D2 is the following:

A stirred solution of DIPA (7.84 ml, 56.00 mmol) in THF (70 ml) was cooled to 0° C. and n-BuLi (35.70 ml of a 1.6 M solution in Cy, 57.10 mmol) was added dropwise. To a solution of dibromomethane (3.58 ml, 51.30 mmol) in THF (70 ml) cooled to −90° C. was added dropwise the LDA solution previously prepared. After 5 min stirring, a solution of 1,1-dimethylethyl (2S)-2-[2-(methyloxy)-2-oxoethyl]-1-piperidinecarboxylate D1 (6.00 g, 23.30 mmol) in THF (47 ml) was added dropwise to the reaction mixture and then, after 10 min, n-BuLi (22.20 ml of a 1.6 M solution in Cy, 35.50 mmol) was added. After 5 min the resulting mixture was added, via cannula, to a rapidly stirring solution of AcCl (35.00 ml, 492 mmol) in absolute EtOH (230 ml) cooled to −78° C. The reaction mixture was left under stirring and then diluted with Et₂O (400 ml). The mixture was transferred into a separatory funnel and washed with a cold 10% H₂SO₄ aqueous solution (2×100 ml), a 5% NaHCO₃ aqueous solution (100 ml) and brine (100 ml). The organic phase was dried (Na₂SO₄), filtered and the solvent removed under reduced pressure. Purification by flash chromatography on silica gel (Biotage SP1 40 M, DCM) gave the title compound D2 (1.14 g, 3.56 mmol, 15% yield). NMR and MS confirmed the product.

Alternative Preparation (iii)

In a 1 L round-bottom flask titanocene dichloride (60 g, 0.24 mol) was suspended in dry toluene (300 ml) under nitrogen atmosphere and cooled down to 0° C. Methylmagnesium chloride (3 M solution in THF, 180 ml, 0.54 mol) was added dropwise (over 45 min), keeping the internal temperature below 8° C. The resulting mixture was stirred at 0-5° C. for 1.5 h and then transferred (over 30 min) through a siphon in an ice-cooled 6% w/w NH₄Cl aqueous solution (180 ml), keeping the internal temperature below 5° C. The mixture was stirred at 0-5° C. for 1 h. Celite (15 g) was added, the mixture stirred at 10° C. for 15 min and then filtered washing with toluene (20 ml). Phases were separated. The organic layer was washed with water (180 ml) and brine (180 ml), dried (Na₂SO₄), filtered and then distilled down under vacuo to 200 ml. The dimethyltitanocene solution in toluene was charged in a 1 L round-bottom flask under nitrogen atmosphere and 1,1-dimethylethyl (2S)-2-[2-(methyloxy)-2-oxoethyl]-1-piperidinecarboxylate (20 g, 0.078 mol) was added. The resulting mixture was stirred at 90° C. for 3 h. Toluene (500 ml) and iso-octane (500 ml) were added and the mixture filtered through a celite pad to remove inorganic salts. A CUNO filtration (R55S cartridge) was then performed to remove the finest particle size solid. The resulting clear solution was concentrated under vacuo to afford the intermediate 1,1-dimethylethyl (2S)-2-{2-[(methyloxy)methyl]-2-propen-1-yl}-1-piperidinecarboxylate as an orange oil (13.60 g, 0.053 mol, 68% yield). HPLC (walk-up): rt=4.69 min. ¹H-NMR (400 MHz, CDCl₃) δ(ppm): 4.42-4.58 (m, 1 H), 3.94-4.08 (m, 1 H), 3.88-3.93 (m, 2 H), 3.53 (s, 3 H), 2.79 (t, 1 H), 2.42 (dd, 1H), 2.27 (dd, 1 H), 1.50-1.70 (m, 6 H), 1.46 (s, 9 H). NBS (8.36 g, 0.047 mol) was added portionwise to a mixture of 1,1-dimethylethyl (2S)-2-{2-[(methyloxy)methyl]-2-propen-l-y1}-1-piperidinecarboxylate (10 g, 0.039 mol) in THF (70 ml) and H₂O (15 ml). The mixture was diluted with TBME (100 ml) and water (50 ml). The aqueous phase was back-extracted with TBME (50 ml). The collected organic phases were washed (twice) with a 4% w/w NaHCO₃ aqueous solution, dried (Na₂SO₄), filtered and evaporated under vacuo. The residual oil was purified by filtration through a silica pad (20 g, toluene/EtOAc 90/10). A further filtration through a silica pad (50 g, toluene/TBME 90/10) afforded the title compound D2 (7.80 g, 0.024 mol, 62% yield).

¹H-NMR (600 MHz, DMSO-d₆) δ(ppm): 4.50-4.64 (m, 1 H), 4.35 (s, 2 H), 3.70-3.88 (m, 1 H), 2.86-3.01 (m, 1 H), 2.65-2.82 (m, 2 H), 1.42-1.60 (m, 5 H), 1.35 (s, 9 H), 1.14-1.28 (m, 1 H).

### Description 3

### 6-chloro-5-methyl-4-pyrimidinamine (D3):

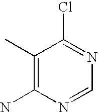

A mixture of 4,6-dichloro-5-methylpyrimidine (available from Sigma-Aldrich #595446) (5 g, 30.70 mmol) in ammonia (7 M solution in MeOH, 15 ml, 105 mmol) was left under stirring for 40 min in a sealed vial at 140° C. Water (300 ml) and EtOAc (600 ml) were added to the resulting white suspension and the two layers were separated. The aqueous phase was extracted with EtOAc (3×600 ml). The collected organic phases were dried (Na₂SO₄), filtered and concentrated under reduced pressure to give the title compound D3 (3.10 g, 20.73 mmol, 68% yield) as a white solid. UPLC: rt=0.41 min, peaks observed: 144 (M+1, 100%) and 146 (M+1, 33%). C₅H₆ClN₃ requires 143. ¹H NMR (400 MHz, DMSO-d₆) δ(ppm): 8.06 (s, 1 H), 7.09 (bs, 2 H), 2.07 (s, 3 H).

### Description 4

### 7-chloro-8-methyl-2-[(2S)-2-piperidinylmethyl]imidazo[1,2-c]pyrimidine (D4):

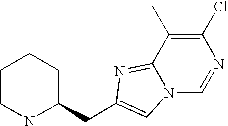

To a solution of 1,1-dimethylethyl (2S)-2-(3-bromo-2-oxopropyl)-1-piperidinecarboxylate D2 (3 g, 9.37 mmol) in DMF (25 ml) was added 6-chloro-5-methyl-4-pyrimidinamine D3 (1.48 g, 10.31 mmol) and the resulting mixture was stirred at 80° C. for 4 h. The solvent was removed under reduced pressure and Et₂O (300 ml) and water (200 ml) were added to the residue. The two layers were separated and the organic one was back-extracted with DCM (2×200 ml). The collected organic phases were dried (Na₂SO₄) and evaporated under reduced pressure. The residue was purified by flash chromatography on silica gel (Biotage SP4 40 M, from DCM 100 to DCM/MeOH 95/5). Collected fractions gave a solid that was eluted through a SCX column to afford a crude material (1 g) containing the title compound D4, the corresponding N-Boc protected derivative and some residual 6-chloro-5-methyl-4-pyrimidinamine [N-Boc derivative data. UPLC: rt=0.74 min, peaks observed: 365 (M+1, 100%) and 367 (M+1, 33%). C₁₈H₂₅ClN₄O₂ requires 364]. The crude was dissolved in DCM (10 ml) and the resulting solution cooled down to 0° C. TFA (2.50 ml) was added and the reaction left under stirring for 4 h at room temperature. Volatiles were removed under reduced pressure and the residue eluted through a SCX column (50 g) to afford the title compound D4 (0.80 g) contaminated with some residual 6-chloro-5-methyl-4-pyrimidinamine HPLC (walk-up): rt=2.44 min. UPLC: rt=0.41 min, peaks observed: 265 (M+1, 100%) and 267 (M+1, 33%). C₁₃H₁₇ClN₄ requires 264.

### Description 5

### 5-methyl-6-(methyloxy)-4-pyrimidinamine (D5):

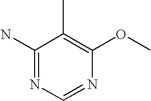

To a suspension of 6-amino-5-methyl-4(1H)-pyrimidinone (available from Chemstep #19148) (1 g, 7.99 mmol) in THF (60 ml) was added triphenylphosphine (4.19 g, 15.98 mmol). The mixture was degassed (3×pump/N₂) and then MeOH (0.65 ml, 15.98 mmol) was added. The resulting reaction mixture was cooled down to 0° C. and DIAD (3.26 ml, 16.78 mmol) was added dropwise. The mixture was allowed to warm up to room temperature, left under stirring for 30 min and concentrated under reduced pressure. The resulting yellow oil was eluted through a SCX column (70 g) and then further purified by flash chromatography on silica gel (Biotage SP4 40 M, from DCM 100 to DCM/MeOH 95/5). Collected fractions gave the title compound D5 (0.31 g, 2.22 mmol, 28% yield) as a white solid. UPLC: rt=0.33 min, peak observed: 140 (M+1). C₆H₉N₃O requires 139. ¹H NMR (400 MHz, DMSO-d₆) δ(ppm): 7.97 (s, 1 H), 6.36 (bs, 2 H), 3.79 (s, 3 H), 1.83 (s, 3 H).

### Description 6

### 8-methyl-7-(methyloxy)-2-[(2S)-2-piperidinylmethyl]imidazo[1,2-c]pyrimidine (D6):

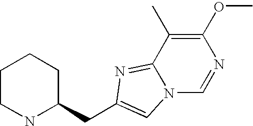

To a solution of 1,1-dimethylethyl (25)-2-(3-bromo-2-oxopropyl)-1-piperidinecarboxylate D2 (0.15 g, 0.49 mmol) in DMF (2 ml) was added 5-methyl-6-(methyloxy)-4-pyrimidinamine D5 (0.081 g, 0.59 mmol) and the reaction mixture was stirred at 80° C. for 45 min. The mixture was then eluted through a SCX column (10 g). Collected fractions gave an oil (0.19 g) containing the title compound D6, the corresponding N-Boc protected derivative and some residual 5-methyl-6-(methyloxy)-4-pyrimidinamine. [N-Boc derivative data. UPLC: rt=0.58 min, peak observed: 361 (M+1). C₁₉H₂₈N₄O₃ requires 360]. The crude was dissolved in DCM (2 ml) and the resulting solution cooled down to 0° C. TFA (0.40 ml) was added dropwise and the reaction left under stirring for 1 h. Volatiles were removed under reduced pressure and the residue eluted through a SCX column (5 g) to afford the title compound D6 (0.16 g) contaminated with some residual 5-methyl-6-(methyloxy)-4-pyrimidinamine UPLC: rt=0.34 min, peak observed: 261 (M+1). C₁₄H₂O N₄O requires 260.

### Description 7

### 8-chloro-7-methyl-2-[(2S)-2-piperidinylmethyl]imidazo[1,2-c]pyrimidine (D7):

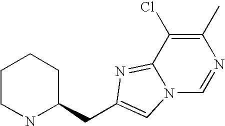

To a solution of 1,1-dimethylethyl (2S)-2-(3-bromo-2-oxopropyl)-1-piperidinecarboxylate D2 (0.10 g, 0.31 mmol) in DMF (1.50 ml) was added 5-chloro-6-methyl-4-pyrimidinamine (available from Chemstep #19785) (0.049 g, 0.34 mmol) and the reaction mixture was stirred at 80° C. for 4 h. The solvent was removed under reduced pressure and the residue was taken-up in Et₂O. The organic phase was washed with water, dried (Na₂SO₄), filtered and evaporated under reduced pressure. The residue was eluted through a SCX column to afford a crude material (0.021 g) containing the title compound D7, the corresponding N-Boc protected derivative and some residual 5-chloro-6-methyl-4-pyrimidinamine. [N-Boc derivative data. UPLC: rt=0.71 min, peaks observed: 365 (M+1, 100%) and 367 (M+1, 33%). C₁₈H₂₅ClN₄O₂ requires 364]. The crude was dissolved in DCM (1 ml) and TFA (2 ml) was added dropwise. The reaction was left under stirring for 1 h at room temperature. Volatiles were removed under reduced pressure and the residue eluted through a SCX column (500 mg) to afford the title compound D7 (0.018 g)contaminated with some residual 5-chloro-6-methyl-4-pyrimidinamine. UPLC: rt=0.40 min, peaks observed: 265 (M+1, 100%) and 267 (M+1, 33%). C₁₃H₁₇ClN₄ requires 264.

### Description 8

### 4-chloro-5,6-dimethylpyrimidine (D8):

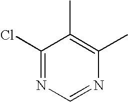

To a mixture of 4,6-dichloro-5-methylpyrimidine (available from Sigma-Aldrich #595446) (0.50 g, 3.07 mmol) and tetrakis(triphenylphosphine)palladium (0) (0.18 g, 0.15 mmol) in toluene (6 ml) and DMF (1 ml) was added cesium carbonate (3 g, 9.20 mmol), followed by methylboronic acid (0.20 g, 3.37 mmol). The resulting reaction mixture was heated at 140° C. under microwave irradiation (2 cycles×30 min), allowed to cool down to room temperature and filtered. Solvents were removed under reduced pressure. The residue was taken-up in EtOAc and washed with a saturated NaHCO₃ aqueous solution and brine. The organic phase was separated, dried (Na₂SO₄), filtered and concentrated to afford the title compound D8 (0.18 g, 1.26 mmol, 41% yield). UPLC: rt=0.53 min, peak observed: 143 (M+1-HCl, 100%) and 445 (M+1-HCl, 33%). C₆H₇ClN₂ requires 142. ¹H NMR (400 MHz, CDCl₃) δ(ppm): 8.70 (s, 1 H), 2.57 (s, 3 H), 2.39 (s, 3 H).

### Description 9

### 5,6-dimethyl-4-pyrimidinamine (D9):

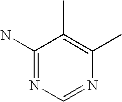

To a solution of 4-chloro-5,6-dimethylpyrimidine D8 (0.18 g, 1.26 mmol) in dry toluene (4 ml) were added sodium t-butoxyde (0.17 g, 1.77 mmol), Pd₂(dba)₃ (0.12 g, 0.13 mmol), BINAP (0.24 g, 0.38 mmol) and benzophenone imine (0.25 ml, 1.51 mmol). The resulting mixture was degassed (3p33 pump/N₂) and then heated to 80° C. After 1 h stirring, the mixture was cooled down to room temperature, diluted with Et₂O (100 ml) and filtered through a celite pad. Volatiles were evaporated, the resulting oil was dissolved in THF (20 ml) and HCl (3 M aqueous solution, 0.63 ml, 1.89 mmol) was added. The mixture was stirred at room temperature for 3 h, concentrated under reduced pressure, neutralized with a saturated NaHCO₃ aqueous solution and diluted with DCM (50 ml). The inorganic layer was back-extracted with DCM (2×50 ml). The collected organic layers were passed through a phase separator tube and evaporated. The orange solid residue was triturated several times with Et₂O and dried to afford the title compound D9 (0.067 g, 0.54 mmol, 42% yield). ¹H NMR (400 MHz, CDCl₃) δ(ppm): 8.38 (s, 1 H), 4.78 (bs, 1 H), 2.43 (s, 3 H), 2.08 (s, 3 H).

### Description 10

### 7,8-dimethyl-2-[(2S)-2-piperidinylmethyl]imidazo[1,2-c]pyrimidine (D10):

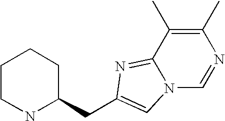

A solution of 1,1-dimethylethyl (2S)-2-(3-bromo-2-oxopropyl)-1-piperidinecarboxylate D2 (0.10 g, 0.31 mmol) and 5,6-dimethyl-4-pyrimidinamine D9 (0.042 g, 0.34 mmol) in DMF (1 ml) was stirred at 80° C. for 4 h. The solvent was evaporated under reduced pressure and the residue purified by flash chromatography on silica gel (from DCM 100 to DCM/MeOH 98/2) and then eluted through a SCX column to afford a crude material (0.018 g) containing the title compound D10, the corresponding N-Boc protected derivative and some residual 5,6-dimethyl-4-pyrimidinamine [N-Boc derivative data. UPLC: rt=0.58 min, peak observed: 345 (M+1). C₁₉H₂₈N₄O₂ requires 344]. The crude was dissolved in DCM (2 ml), TFA (0.50 ml) was added dropwise and the reaction left under stirring for 1 h at room temperature. Volatiles were removed under reduced pressure and the residue eluted through a SCX cartridge to afford a crude material containing the title compound D10 (0.012 g) contaminated with some residual 5,6-dimethyl-4-pyrimidinamine. UPLC: rt=0.36 min, peak observed: 245 (M+1). C₁₄H₂O N₄ requires 244.

### EXAMPLES

### Example 1

### 7-chloro-8-methyl-2-({2S)-1-[(2-methyl-5-phenyl-1,3-thiazol-4-ylcarbonyl]-2-piperidinyl}methyl)imidazo[1,2-c]pyrimidine (HCl salt) (E1):

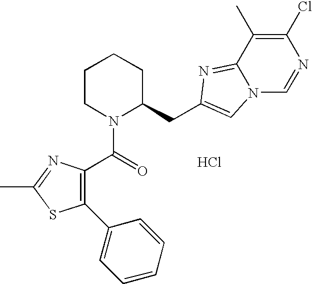

To a solution of 2-methyl-5-phenyl-1,3-thiazole-4-carboxylic acid (0.73 g, 3.32 mmol) in DCM (20 ml), oxalyl chloride (0.58 ml, 6.65 mmol) and DMF (0.050 ml) were added and the resulting mixture stirred at room temperature for 30 min. Volatiles were removed under reduced pressure and the residue was dissolved in DCM (15 ml). The acyl chloride solution was added dropwise at 0° C. to a mixture of 7-chloro-8-methyl-2-[(2S)-2-piperidinylmethyl]imidazo[1,2-c]pyrimidine D4 (0.80 g of the crude material obtained in Description 4) and TEA (1.26 ml, 9.07 mmol) in DCM (20 ml) and the mixture was stirred at room temperature for 2 h. The reaction mixture was then diluted with DCM (60 ml) and washed with a saturated NaHCO₃ aqueous solution (3×80 ml) and brine (80 ml). The organic phase was collected by a phase separator tube and concentrated. The residue was purified by flash chromatography on silica gel (Biotage SP1 40 M, from DCM 100 to DCM/MeOH 97/3). Collected fractions gave the free base of the title compound E1 (1.04 g, 2.23 mmol, 24% yield from D2, three steps).

HPLC (walk-up): rt=4.09 min. UPLC: rt=0.70 min, peaks observed: 466 (M+1, 100%) and 468 (M+1, 33%). C₂₄H₂₄ClN₅OS requires 465. ¹H NMR [the product is present as a mixture of conformers (ratio ca. 65/35) and the assignment refers to the major component] (500 MHz, DMSO-d₆) δ(ppm): 9.05 (s, 1 H), 7.67 (s, 1 H), 7.13-7.21 (m, 3 H), 7.02-7.09 (m, 2 H), 4.45 (dd, 1 H), 4.01-4.10 (m, 1 H), 3.09-3.26 (m, 2 H), 3.04 (t, 1 H), 2.47 (s, 3 H), 2.21 (s, 3 H), 0.80-1.79 (m, 6 H). The free base (1.04 g, 2.23 mmol) was dissolved in DCM (10 ml) and Et₂O (25 ml), the solution was cooled down to 0° C. and a 1 M HCl solution in Et₂O (5 ml, 5 mmol) was added dropwise. The mixture was left under stirring at room temperature for 10 min. Volatiles were evaporated under reduced pressure and the residue was triturated several times with cold Et₂O to afford the title compound E1 (1.12 g, 2.19 mmol, 98% yield). HPLC (walk-up): rt=4.09 min. UPLC: rt=0.70 min, peaks observed: 466 (M+1-HCl, 100%) and 468 (M+1-HCl, 33%). C₂₄H₂₅Cl₂N₅OS requires 501.

### Example 2

### 8-methyl-2-({2S)-1-[(2-methyl-5-phenyl-1,3-thiazol-4-yl)carbonyl]-2-piperidinyl}tmethyl)imidazo[1,2-c]pyrimidine (HCl salt) (E2):

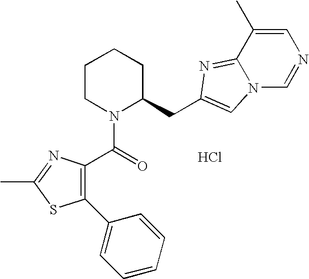

To a solution of 7-chloro-8-methyl-2-({(2S)-1-[(2-methyl-5-phenyl-1,3-thiazol-4-yl)carbonyl]-2-piperidinyl}methyl)imidazo[1,2-c]pyrimidine hydrochloride E1 (13.50 mg, 0.027 mmol) in MeOH (1 ml) was added Pd/C (1.43 mg, 0.013 mmol), followed by ammonium formate (5.79 mg, 0.092 mmol). The reaction mixture was left under stirring at room temperature for 1 h and then at 40° C. for 5 h. After a further addition of Pd/C (1 eq) and ammonium formate (3 eq) the mixture was stirred at 80° C. for 10 h, cooled down to room temperature and eluted through a SCX column. Collected fractions gave a crude material that was purified by flash chromatography on silica gel (5 g cartridge, from DCM 100 to DCM/MeOH 90/10) to afford the free base of the title compound E2 (7.50 mg, 0.017 mmol, 65% yield). MS: (ES/+) m/z: 432 (M+1). C₂₄H₂₅N₅OS requires 431. UPLC: rt=0.58 min, peak observed: 432 (M+1). The free base (6.50 mg, 0.015 mmol) was dissolved in DCM (0.50 ml) and Et₂O (0.50 ml) and a 2 M HCl solution in Et₂O (0.30 ml, 0.600 mmol) was added. Volatiles were removed under reduced pressure and the residue was triturated several times with Et₂O to give the title compound E2 (5.50 mg, 0.012 mmol, 78% yield). HPLC (walk-up): rt=3.48 min. MS: (ES/+) m/z: 432 (M+1-HCl). C₂₄H₂₆N₅OSCl requires 467.

### Example 3

### 8-methyl-7-(methyloxy)-2-({(2-S)-1-[(2-methyl-5-phenyl-1,3-thiazol-4-yl)carbonyl]-2-piperidinyl}methyl)imidazo[1,2-c]pyrimidine (HCl salt) (E3):

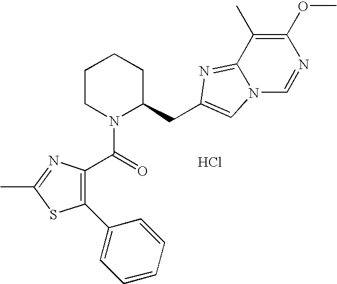

To a solution of 2-methyl-5-phenyl-1,3-thiazole-4-carboxylic acid (35.20 mg, 0.16 mmol) in DCM (1 ml), oxalyl chloride (0.014 ml, 0.16 mmol) and DMF (0.011 ml) were added and the resulting mixture stirred at room temperature for 30 min. Volatiles were removed under reduced pressure and the residue was dissolved in DCM (1 ml). The acyl chloride solution was added dropwise at 0° C. to a mixture of 8-methyl-7-(methyloxy)-2-[(2S)-2-piperidinylmethyl]imidazo[1,2-c]pyrimidine D6 (38 mg of the crude material obtained in Description 6) and TEA (0.061 ml, 0.44 mmol) in DCM (1 ml) and the mixture was stirred at room temperature for 1 h. The reaction mixture was then diluted with DCM (10 ml) and washed with a saturated NaHCO₃ aqueous solution (4 ml). The organic phase was separated, dried (Na₂SO₄), filtered and concentrated. The residue was purified by flash chromatography on NH (Biotage SP4 12 M, Cy/EtOAc from 95/5 to 60/40). Collected fractions gave the free base of the title compound E3 (41 mg, 0.088 mmol, 75% yield from D2, three steps) as a white solid. HPLC (walk-up): rt=3.93 min. MS: (ES/+) m/z: 462 (M+1). C₂₅H₂₇N₅O₂S requires 461. ¹H NMR (500 MHz, DMSO-d₆): δ(ppm): 8.97 (s, 1 H), 7.49 (s, 1 H), 7.03-7.12 (m, 2 H), 7.13-7.24 (m, 3 H), 4.44 (dd, 1 H), 4.04-4.11 (m, 1 H), 3.86 (s, 3 H), 3.10 (dd, 1 H), 3.00 (t, 1 H), 2.63 (dd, 1 H), 2.48 (s, 3 H), 2.03 (s, 3 H), 1.23-1.72 (m, 5 H), 0.82-0.96 (m, 1 H). The free base (39 mg, 0.084 mmol) was dissolved in DCM (1 ml), the solution was cooled down to 0° C. and a 1 M HCl solution in Et₂O (0.13 ml, 0.13 mmol) was added. The mixture was left under stirring for 15 min. Volatiles were evaporated under reduced pressure to afford the title compound E3 (42 mg, 0.083 mmol, 94% yield) as a white solid. HPLC (walk-up): rt=3.98 min. MS: (ES/+) m/z: 462 (M+1-HCl). C₂₅H₂₈ClN₅O₂S requires 497.

### Example 4

### 8-chloro-7-methyl-2-({(2S)-1-[(2-methyl-5-phenyl-1,3-thiazol-4-yl)carbonyl]-2-piperidinyl}methyl)imidazo[1,2-c]pyrimidine (HCl salt) (E4):

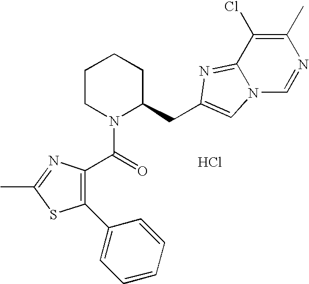

To a solution of 2-methyl-5-phenyl-1,3-thiazole-4-carboxylic acid (16.58 mg, 0.076 mmol) in DCM (1 ml), oxalyl chloride (0.006 ml, 0.076 mmol) and DMF (0.005 ml) were added and the resulting mixture stirred at room temperature for 30 min. Volatiles were removed under reduced pressure and the residue was dissolved in DCM (1 ml). The acyl chloride solution was added dropwise at 0° C. to a mixture of 8-chloro-7-methyl-2-[(2S)-2-piperidinylmethyl]imidazo[1,2-c]pyrimidine D7 (18 mg of the crude material obtained in Description 7) and TEA (0.029 ml, 0.206 mmol) in DCM (1 ml) and the mixture was stirred at room temperature for 1 h. The reaction mixture was then diluted with DCM (15 ml) and washed with a saturated NaHCO₃ aqueous solution (4 ml). The organic phase was separated, dried (Na₂SO₄), filtered and concentrated. The residue was purified by flash chromatography on silica gel (Biotage 12 M, from DCM 100 to DCM/MeOH 98/2). Collected fractions gave the free base of the title compound E4 (16.20 mg, 0.027 mmol, 9% yield from D2, three steps) as a pale yellow solid.

UPLC: rt=0.68 min, peaks observed: 466 (M+1, 100%) and 468 (M+1, 33%).

C₂₄H₂₄ClN₅OS requires 465. ¹H NMR [the product is present as a mixture of conformers (ratio ca. 65/35). The assignment refers to the major component] (500 MHz, DMSO-d₆): δ(ppm): 9.12 (s, 1 H), 7.66 (s, 1 H), 7.16-7.27 (m, 2 H), 7.07-7.15 (m, 3 H), 4.44 (dd, 1 H), 3.92-4.05 (m, 1 H), 3.08-3.25 (m, 1 H), 3.04 (t, 1 H), 2.61-2.67 (m, 1 H), 2.44 (s, 3 H), 2.41 (s, 3 H), 0.75-1.76 (m, 6 H). The free base (15 mg, 0.032 mmol) was dissolved in anhydrous DCM (1 ml), the solution was cooled down to 0° C. and a 1 M HCl solution in Et₂O (0.10 ml, 0.100 mmol) was added. The mixture was left under stirring for 15 min. Volatiles were evaporated under reduced pressure to afford the title compound E4 (17 mg, 0.027 mmol, quantitative yield) as a light brown solid. HPLC (walk-up): rt=4.61 min. MS: (ES/+) m/z: 466 (M+1-HCl). C₂₅H₂₅Cl₂N₅OS requires 501.

### Example 5

### 7,8-dimethyl-2-({(2S)-1-[(2-methyl-5-phenyl-1,3-thiazol-4-yl)carbonyl]-2-piperidinyl}methyl)imidazo[1,2-c]pyrimidine (HCl salt) (E5):

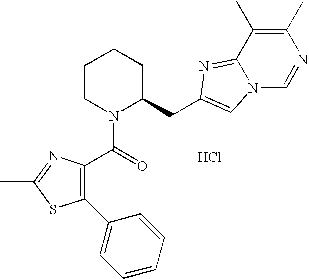

To a solution of 5-phenyl-2-methyl-1,3-thiazole-4-carboxylic acid (16.50 mg, 0.074 mmol) in DMF (2 ml), DIPEA (0.051 ml, 0.29 mmol) and TBTU (26.80 mg, 0.083 mmol) were added and the mixture was left under stirring at room temperature for 30 min. A solution of 7,8-dimethyl-2-[(2S)-2-piperidinylmethyl]imidazo[1,2-c]pyrimidine D10 (0.012 g of the crude material obtained in Description 10) in DMF (1 ml) was added at 0° C. to the activated carboxylic acid and the reaction mixture stirred for 2 h. The mixture was diluted with DCM and washed with a 1 M NaOH aqueous solution and brine. The organic layer was separated, dried (Na₂SO₄) and the solvent removed under reduced. The residue was purified by flash chromatography (from DCM 100 to DCM/MeOH 90/10). Collected fractions gave the free base of the title compound E5 (14 mg, 0.031 mmol, 10% yield from D2, three steps). UPLC rt=0.57 min, peak observed: 446 (M+1). C₂₅H₂₇N₅OS requires 445. The free base (14 mg, 0.031 mmol) was dissolved in DCM (0.50 ml) and Et₂O (0.50 ml) and a 1 M HCl solution in Et₂O (0.031 ml, 0.031 mmol) was added. The mixture was left under stirring for 10 min. Volatiles were evaporated under reduced pressure and the residue was triturated several times with Et₂O to afford the title compound E5 (12.30 mg, 0.023 mmol, 74% yield). UPLC: rt=0.57 min, peak observed: 446 (M+1-HC1). C₂₅H₂₈ClN₅OS requires 481. ¹H NMR [the product is present as a mixture of conformers (ratio ca. 70/30). The assignment refers to the major component] (500 MHz, DMSO-d₆): δ(ppm): 9.42 (s, 1 H), 8.02 (s, 1 H), 6.71-7.63 (m, 5 H), 4.45 (dd, 1 H), 3.72-4.35 (m, 1 H), 3.64 (t, 1 H), 3.04-3.26 (m, 2 H), 2.27-2.73 (m, 3 H), 0.71-0.80 (m, 6 H).

### Example 6

### Determination of ANTAGONIST AFFINITY at Human Orexin-1 and 2 Receptors Using FLIPR

### Cell Culture

Adherent Chinese Hamster Ovary (CHO) cells, stably expressing the recombinant human Orexin-1 or human Orexin-2 receptors or Rat Basophilic Leukaemia Cells (RBL) stably expressing recombinant rat Orexin-1 or rat Orexin-2 receptors were maintained in culture in Alpha Minimum Essential Medium (Gibco/Invitrogen, cat. no.; 22571-020), supplemented with 10% decomplemented foetal bovine serum (Life Technologies, cat. no. 10106-078) and 400 μg/mL Geneticin G418 (Calbiochem, cat. no.345810). Cells were grown as monolayers under 95%:5% air:CO₂ at 37° C.

The sequences of the human orexin 1, human orexin 2, rat orexin 1 and rat orexin 2 receptors used in this example were as published in Sakurai, T. et al (1998) Cell, 92 pp 573 to 585, with the exception that the human orexin 1 receptor sequence used had the amino acid residue alanine at position 280 and not glycine as reported in Sakurai et al.

### Measurement of [Ca^{2+]}ᵢ Using the FLIPR™

Cells were seeded into black clear-bottom 384-well plates (density of 20,000 cells per well) in culture medium as described above and maintained overnight (95%:5% air:CO₂ at 37° C.). On the day of the experiment, culture medium were discarded and the cells washed three times with standard buffer (NaCl, 145 mM; KCl, 5 mM; HEPES, 20 mM; Glucose, 5.5 mM; MgCl₂, 1 mM; CaCl₂, 2 mM) added with Probenecid 2.5 mM. The plates were then incubated at 37° C. for 60 minutes in the dark with 1 μM FLUO-4AM dye to allow cell uptake of the FLUO-4AM, which is subsequently converted by intracellular esterases to FLUO-4, which is unable to leave the cells. After incubation, cells were washed three times with standard buffer to remove extracellular dye and 30 μL of buffer were left in each well after washing.

Compounds of the invention were tested in a final assay concentration range from 1.66×10⁻⁵M to 1.58×10⁻¹¹M. Compounds of the invention were dissolved in dimethylsulfoxide (DMSO) at a stock concentration of 10 mM. These stock solutions were serially diluted with DMSO and 1 μL of each dilution was transferred to a 384 well compound plate. Immediately before introducing compound to the cells, buffer solution (50 μl/well) was added to this plate. To allow agonist stimulation of the cells, a stock plate containing a solution of human orexin A (hOrexin A) was diluted with buffer to final concentration just before use. This final concentration of hOrexin A was equivalent to the calculated EC80 for hOrexinA agonist potency in this test system. This value was obtained by testing hOrexinA in concentration response curve (at least 16 replicates) the same day of the experiment.

The loaded cells were then incubated for 10min at 37° C. with test compound. The plates were then placed into a FLIPRTM (Molecular Devices, UK) to monitor cell fluorescence (λₑₓ=488 nm, λ_{EM}=540 nm) (Sullivan E, Tucker E M, Dale I L. Measurement of [Ca²⁺]ᵢ, using the fluometric imaging plate reader (FLIPR). In: Lambert D G (ed.), Calcium Signaling Protocols. New Jersey: Humana Press, 1999, 125-136). A baseline fluorescence reading was taken over a 5 to 10 second period, and then 10 μL of EC80 hOrexinA solution was added. The fluorescence was then read over a 4-5 minute period.

### Data Analysis

Functional responses using FLIPR were measured as peak fluorescence intensity minus basal fluorescence and expressed as a percentage of a non-inhibited Orexin-A-induced response on the same plate. Iterative curve-fitting and parameter estimations were carried out using a four parameter logistic model and Microsoft Excel (Bowen W P, Jerman J C. Nonlinear regression using spreadsheets. Trends Pharmacol. Sci. 1995; 16: 413-417). Antagonist affinity values (IC₅₀) were converted to functional pKᵢ values using a modified Cheng-Prusoff correction (Cheng Y C, Prusoff W H. Relationship between the inhibition constant (Kᵢ) and the concentration of inhibitor which causes 50 percent inhibition (IC₅₀) of an enzymatic reaction. Biochem. Pharmacol. 1973, 22: 3099-3108).

$$fpKi = - log \frac{( {IC}_{50} )}{{( 2 + {( \frac{[ agonist ]}{( {EC}_{50} )} )}^{n} )}^{1 / n} - 1}$$

Where [agonist] is the agonist concentration, EC₅₀ is the concentration of agonist giving 50% activity derived from the agonist dose response curve and n=slope of the dose response curve. When n=1 the equation collapses to the more familiar Cheng-Prusoff equation.

Compounds of examples 1 to 5 were tested according to the method of example 6. All compounds gave fpKi values from 8.4 to 9.2 at the human cloned orexin-1 receptor (having the amino acid residue alanine at position 280 and not glycine) and from 8.1 to 9.1 at the human cloned orexin-2 receptor.

1-25. (canceled)

26. A compound of formula (I) 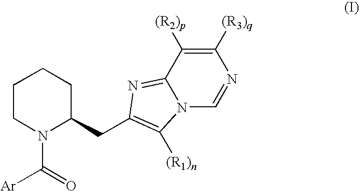
where Ar is selected from: 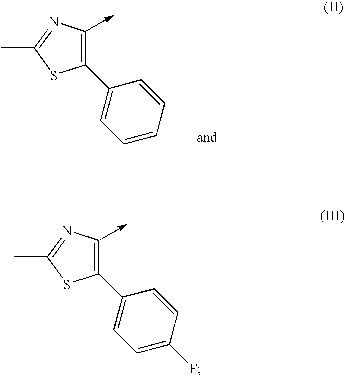
R₁ is (C₁₋₄)alkyl, halo, halo(C₁₋₄alkyl, (C₁₋₄)alkoxy, halo(C₁₋₄alkoxy, (C₁₋₄)alkyl-O-(C₁₋₄)alkyl, CN, NR⁴R⁵wherein R⁴ is H or (C₁₋₄)alkyl and R⁵ is H or (C₁₋₄)alkyl;
R₂ is (C₁₋₄)alkyl, halo, halo(C₁₋₄alkyl, (C₁₋₄)alkoxy, halo(C₁₋₄alkoxy, (C₁₋₄)alkyl-O-(C₁₋₄)alkyl, CN, NR⁶R⁷wherein R⁶ is H or (C₁₋₄-alkyl and R⁷ is H or (C₁₋₄)-alkyl;
R₃ is (C₁₋₄)alkyl, halo, halo(C₁₋₄alkyl, (C₁₋₄)alkoxy, halo(C₁₋₄alkoxy, (C₁₋₄)alkyl-O-(C₁₋₄)alkyl, CN, NR⁸R⁹wherein R⁸ is H or (C₁₋₄-alkyl and R⁹ is H or (C₁₋₄)-alkyl;
n is 0 or 1;
p is 0 or 1; and
q is 0 or 1;
with the proviso that p and q are not both 0;
or a pharmaceutically acceptable salt thereof.

27. The compound or salt according to claim 25, where Ar is a group of formula (II).

28. The compound or salt according to claim 25, where Ar is a group of formula (III).

29. The compound or salt according to claim 25, where n is 0.

30. The compound or salt according to claim 29, where p is 1, q is 0 and R₂ is methyl.

31. The compound or salt according to claim 29, where p is 1, q is 1 and one of R₂ and R₃ is halo and the other is (C₁₋₄)-alkyl.

32. The compound or salt according to claim 31, where R₂ is (C₁₋₄-alkyl and R₃ is halo.

33. The compound or salt according to claim 32, where R₂ is methyl and R₃ is chloro.

34. The compound or salt according to claim 31, where R₂ is halo and R₃ is (C₁₋₄)-alkyl.

35. The compound or salt according to claim 34, where R₂ is chloro and R₃ is methyl.

36. A compound selected from the group consisting of:
7-chloro-8-methyl-2-({(2S)-1-[(2-methyl-5-phenyl-1,3-thiazol-4-yl)carbonyl]-2-pipe ridinyl}methyl)imidazo[1,2-c]pyrimidine;
8-methyl-2-({(2S)-1-[(2-methyl-5-phenyl-1,3-thiazol-4-yl)carbonyl]-2-piperidinyl}methyl)imidazo[1,2-c]pyrimidine;
8-chloro-7-methyl-2-({(2S)-1-[(2-methyl-5-phenyl-1,3-thiazol-4-yl)carbonyl]-2-pipe ridinyl}methyl)imidazo[1,2-c]pyrimidine;
8-methyl-7-(methyloxy)-2-({(2S)-1-[(2-methyl-5-phenyl-1,3-thiazol-4-yl)carbonyl]-2-piperidinyl}methyl)imidazo[1,2-c]pyrimidine; and
7,8-dimethyl-2-({(2S)-1-[(2-methyl-5-phenyl-1,3-thiazol-4-yl)carbonyl]-2-piperidinyl}methyl)imidazo[1,2-c]pyrimidine,
or a pharmaceutically acceptable salt thereof.

37. A method of treating or preventing a disease or disorder where an antagonist of a human orexin receptor is required, which comprises administering to a subject in need thereof an effective amount of the compound or salt according to claim 25, where the disease or disorder is a sleep disorder, a depression or mood disorder, an anxiety disorder, a substance-related disorder or a feeding disorder.

38. The method according to claim 37, where the disease or disorder is a sleep disorder.

39. The method according to claim 38, where the sleep disorder is selected from the group consisting of Primary Insomnia; a Breathing-Related Sleep Disorder; Circadian Rhythm Sleep Disorder; a Dyssomnia Not Otherwise Specified; Nightmare Disorder; Sleep Terror Disorder; Sleepwalking Disorder; a Parasomnia Not Otherwise Specified; Insomnia Related to Another Mental Disorder; a sleep disturbance associated with a disease or disorder selected from a neurological disorder, neuropathic pain, restless leg syndrome, heart disease and lung disease; a Substance-Induced Sleep Disorder subtype selected from Insomnia Type, Parasomnia Type and Mixed Type; Sleep Apnea; and Jet-Lag Syndrome.

40. A pharmaceutical composition comprising a) the compound or salt according to claim 25, and b) a pharmaceutically acceptable carrier.
